(19) United States
(12) Patent Application Publication
(10) Pub. No.: US 2024/0058819 A1
(43) Pub. Date: Feb. 22, 2024

(54) MODULAR WELL PLATE SYSTEM WITH A REUSABLE FRAME

(71) Applicant: Cerillo, Inc., Charlottesville, VA (US)

(72) Inventors: Kevin Seitter, Charlottesville, VA (US); Andrew Homyk, Charlottesville, VA (US); Jeff Ference, JR., Charlottesville, VA (US); John Peck, Charlottesville, VA (US); Jessica Lee, Charlottesville, VA (US); Sydney DeCleene, Charlottesville, VA (US); Derek Boylan, Charlottesville, VA (US); Daniel Carrier, Charlottesville, VA (US)

(21) Appl. No.: 18/452,068

(22) Filed: Aug. 18, 2023

Related U.S. Application Data
(60) Provisional application No. 63/371,762, filed on Aug. 18, 2022.

(51) Int. Cl. B01L 3/00

(52) U.S. Cl. CPC B01L 3/5085; B01L 2300/0829; B01L 2300/163

(57) ABSTRACT

Herein are described modular well plate systems having a reusable frame that is configured to receive a plurality of disposable modules. The systems being useful for, inter alia, performing multiplexed analytical scientific research.

### CROSS REFERENCE TO RELATED APPLICATIONS

This application is entitled to priority pursuant to 35 U.S.C. § 119(e) to U.S. provisional patent application No. 63/371,762, filed on 18 Aug. 2022. The contents of which are incorporated herein by reference in their entirety.

### BACKGROUND OF THE INVENTION

Specialty culturing plates for analytical analysis of microbial (e.g., microbiome) specimens can be expensive to purchase as whole units or plates. Furthermore, the reagents and specimens used in the preparation of these plate samples can be costly or time intensive to prepare in larger volumes.

It is desirable to have a system where researchers can evaluate their systems and methodologies in small batches or quantities to hone their techniques before committing to more costly or time intensive studies that utilize a full-sized plate (or plates).

### SUMMARY OF THE INVENTION

In an aspect, there is described a modular well plate system, comprising a reusable frame configured to temporarily affix a plurality of disposable modular wells (modules).

In another aspect, there is described a modular well plate system, comprising a plurality of disposable modules, each having a plurality of chambers and one or more registration features and a reusable frame comprising horizontal and vertical reference planes, a plurality of receiving areas for the modules, and one or more corresponding registration features.

These and other aspects, which will become apparent during the following detailed description, have been achieved by the inventors' discovery of novel, modular well plate systems.

### BRIEF DESCRIPTION OF THE DRAWINGS

FIGS. 1A and 1B are isometric views of modular well plate systems depicting a reusable frame fully populated with 8 or 18, respectively, co-culturing modules.

FIGS. 2A and 2B are top views of modular well plate systems having reusable frames.

FIGS. 3A and 3B are isometric views of individual modules, a plurality of which, in combination with the frame, make up a well plate system. In FIGS. 3A and 3B, two well volumes are connected together forming an individual co-culture module.

FIGS. 4A and 4B are top views of individual modules each showing a measurement window or opening.

FIGS. 5A and 5B are isometric views depicting the bottoms of individual modules, showing bottom alignment and registration features, as well alignment features.

FIGS. 6A and 6B are isometric views of modular well plate systems having reusable frames. In FIGS. 6A and 6B, several individual modules have been removed from the frames, illustrating their use when partially populated.

FIGS. 7A and 7B are isometrics view of reusable frames.

FIGS. 8A and 8B are top views of reusable frames, showing flexure tabs used to align and register individual modules.

FIG. 9 shows a reusable frame having a logo plague.

### DETAILED DESCRIPTION OF THE INVENTION

Exemplary aspects of the present invention are described herein. Although the following detailed description contains many specifics for purposes of illustration, a person of ordinary skill in the art will appreciate that variations and alterations to the following details are within the scope of the invention. Accordingly, the following aspects of the invention are set forth without any loss of generality to, and without imposing limitations upon, the claimed invention.

An aspect herein involves an apparatus, comprising: a reusable frame configured to temporarily hold a plurality of individual, disposable, microplate modules. In some aspects, these individual, disposable modules, comprise: specialty wells, co-culturing pairs, or other combinations thereof. In some aspects, the modules are designed to fit snugly into the reusable frame forming an assembly that is suited to fit in a variety of microplate readers for scientific examination of samples contained in the modules.

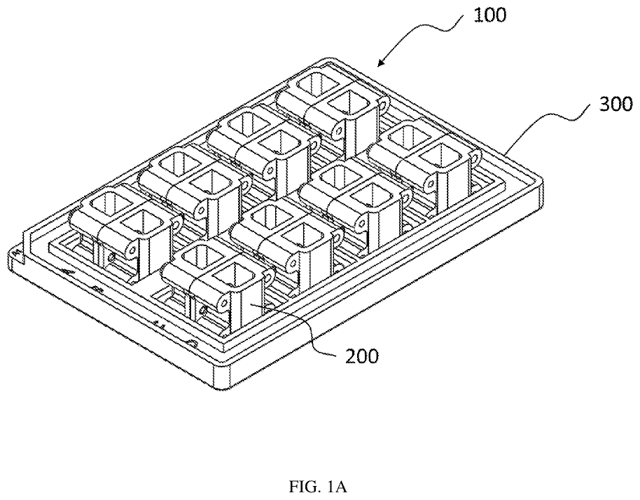

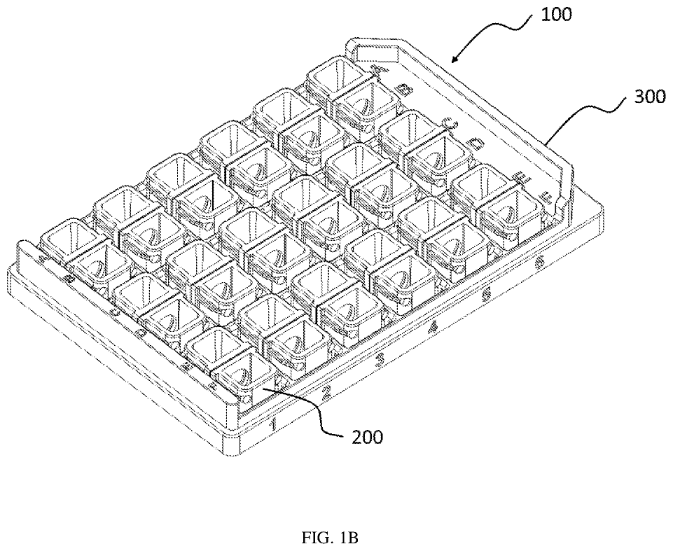

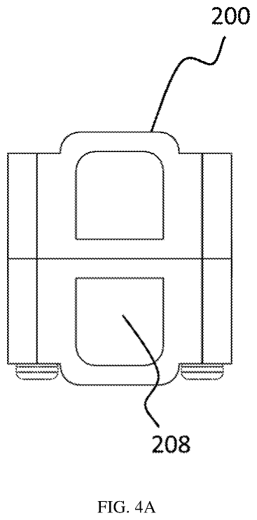

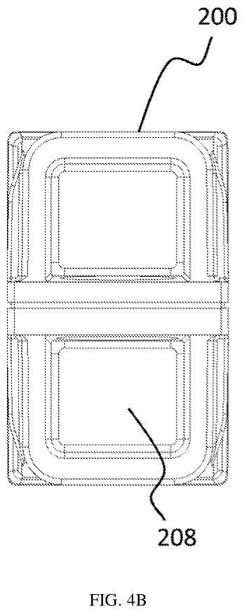

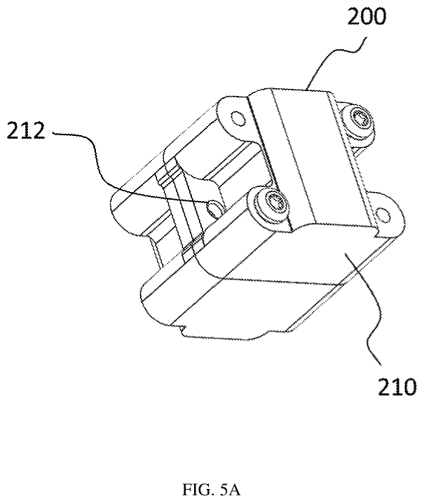

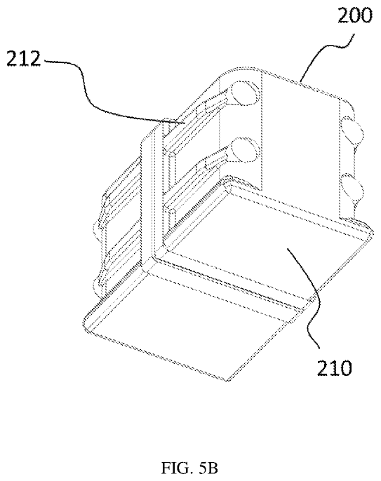

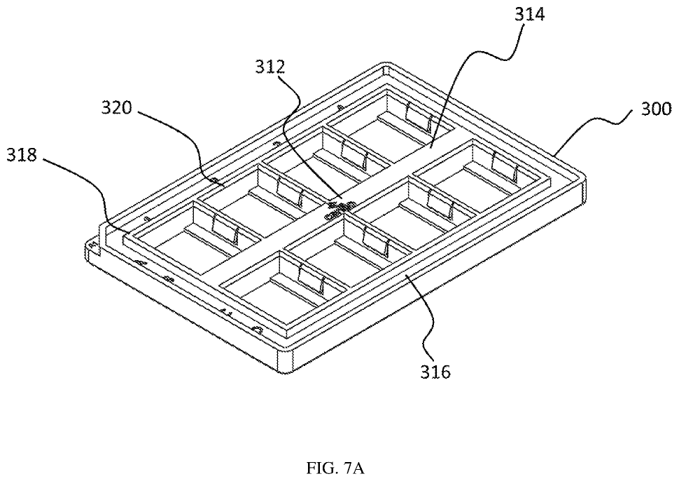

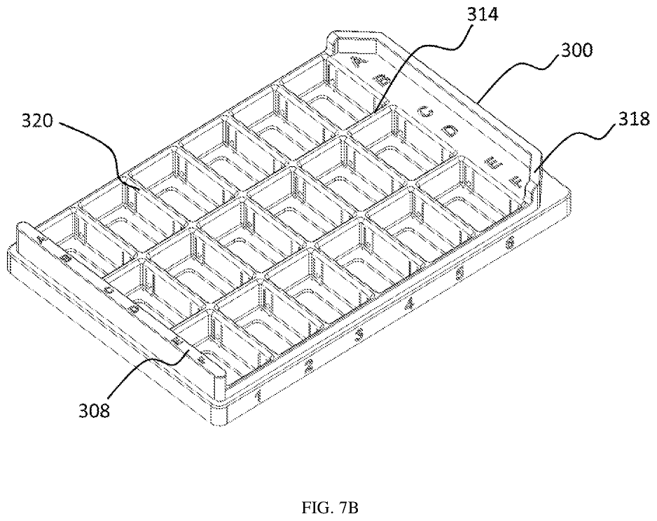

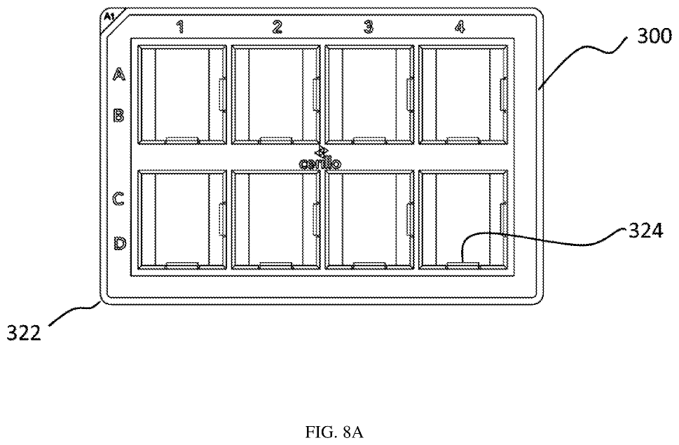

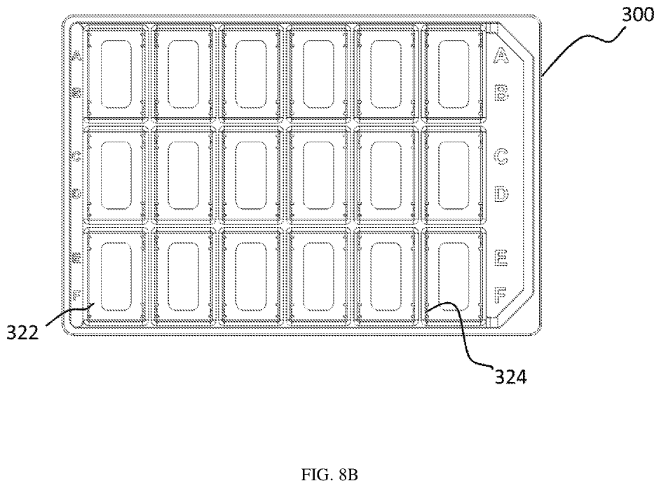

Names for components shown in the FIGS. 1A-9 are as follows:

| | |
|---|---|
| 100 | Plate system with modular wells and a reusable frame |
| 200 | Modular well |
| 300 | Reusable frame |
| 102 | Origin indicator |
| 104 | Horizontal reference plane |
| 106 | Coordinate symbols |
| 108 | Horizontal registration feature for module to frame |
| 110 | Vertical reference plane |
| 112 | Vertical registration feature for module to frame |
| 202 | Module top surface |
| 204 | Module horizontal registration surface |
| 206 | Module vertical registration surface |
| 208 | Module measurement window or opening |
| 210 | Module bottom registration surface |
| 212 | Module alignment feature |
| 302 | Frame measurement window or opening |
| 304 | Frame module support ledge |
| 306 | Frame registration flexure |
| 308 | Frame perimeter ridge |
| 310 | Frame origin cutaway |
| 312 | Frame logo plaque |
| 314 | Frame central support rib |
| 316 | Frame spill cache |
| 318 | Frame interior ridge |
| 320 | Frame module lead-in feature |
| 322 | Rounded frame corner |
| 324 | Frame secondary registration flexure |

Referring to FIGS. 1A and 1B, there is illustrated a modular well plate system 100, comprising: a reusable frame 300 and a plurality of disposable, modular wells 200 (also called modules). FIG. 1A shows 8 modules. FIG. 1B shows 18 modules.

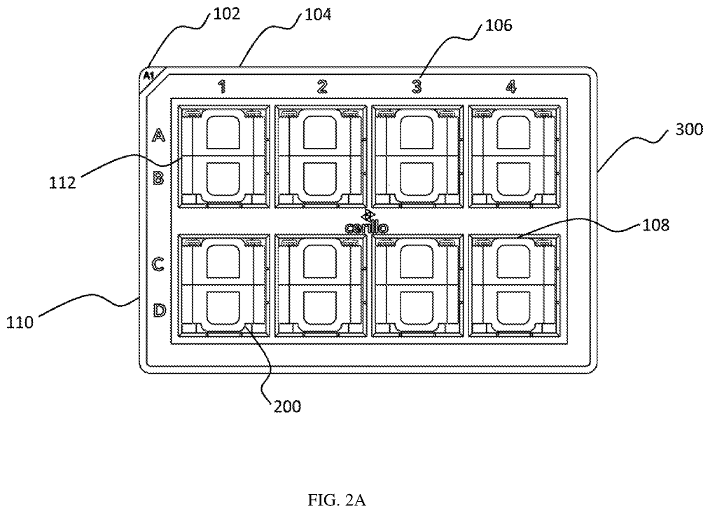

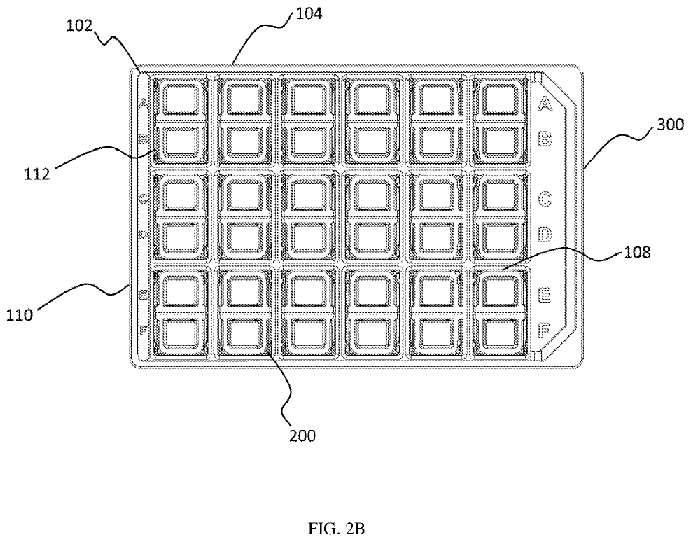

Referring to FIGS. 2A and 2B, there is illustrated a top view of a modular well plate system wherein the position of the reusable frame 300 is aligned via reference planes 104 (horizontal) and 110 (vertical) to a measurement device that is used to measure sample parameters contained within the modular well plate system 100. The frame is shown with an origin indicator 102 that is designed to identify the origin of the reusable frame 300 and a coordinate system 106 that is configured to allow for identification of the plurality of disposable, modular wells 200. The horizontal registration feature 108 and vertical registration feature 112 are configured to temporarily affix the modular wells 200 to frame 300.

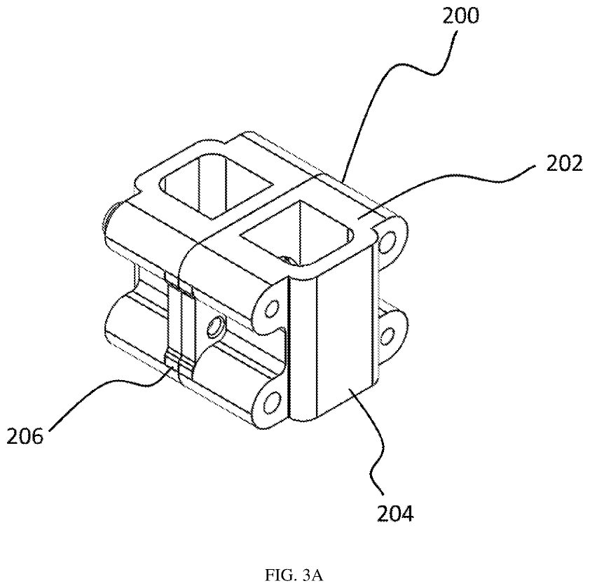

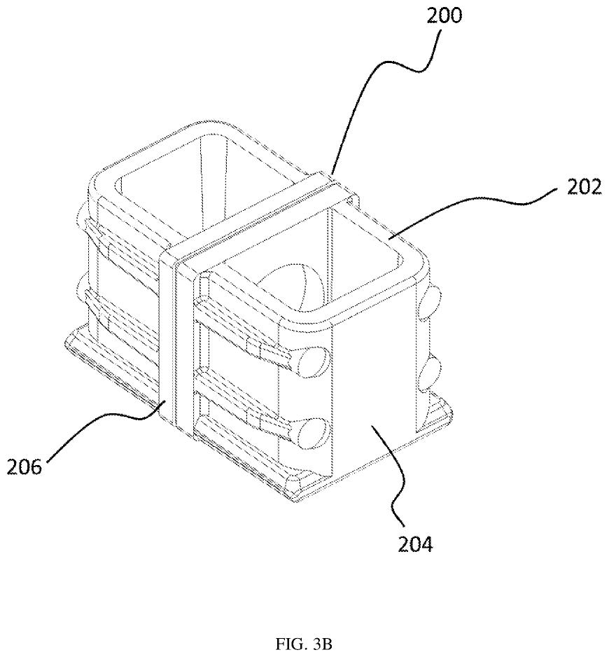

Referring to FIGS. 3A-5B, there is illustrated a disposable, modular well 200 (module). The module has a top surface 202, two chambers (shown, but unnumbered), a horizontal registration surface 204 configured to align the module to a corresponding feature 108 on reusable frame 300 and a vertical registration surface 206 configured to align the module to a corresponding feature 112 on reusable frame 300. The module is also shown having a measurement window or opening 208, bottom registration surface 210, and alignment feature 212.

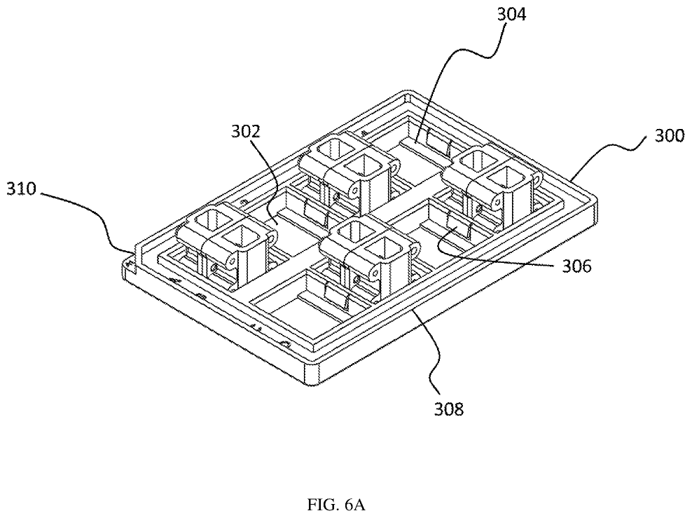

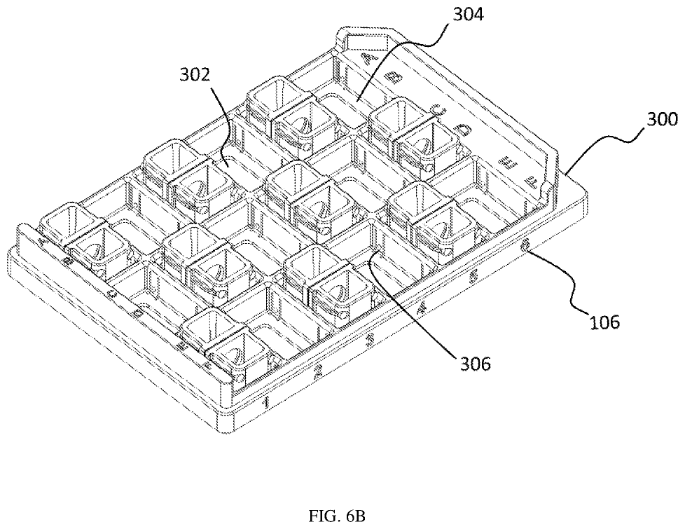

Referring to FIGS. 6A-8A, there is illustrated a reusable frame 300. The frame is shown with a plurality of measurement windows or openings 302, a plurality of module support ledges 304, a plurality of registration flexures 306, a perimeter ridge 308, an origin cutaway 310, a logo plaque 312, a central support rib 314, a spill cache 316, an interior ridge 318, a module lead-in feature 320, rounded frame corners 322, and secondary registration flexures 324.

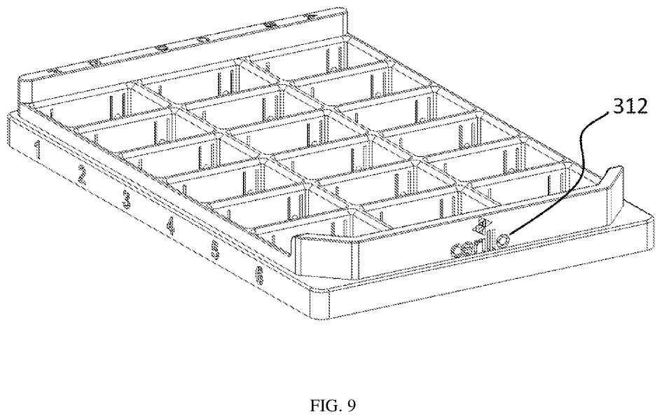

Referring to FIG. 9, there is illustrated an alternative location for logo plaque 312.

The perimeter and alignment features described herein are designed according to ANSI standards for common microplate sizing (e.g., 127.76 mm by 85.48 mm), and the modular well plate system 100 is designed to fit as assembled with an optional lid (not shown) into instruments and devices common to laboratory and diagnostic practice for microplates and analogous products.

Another aspect involves a novel, modular well plate system 100, comprising:
A a reusable frame 300; and,
B one or more modular wells 200;
   wherein the reusable frame 300 is configured to receive and temporarily affix a plurality of modular wells 200 and the modular wells 200 are disposable or, alternatively, reusable.

The reusable frame 300, sometimes called an alignment frame, is configured to securely, but temporarily, fasten and align one or more individual modules 200 to the frame. Once the modules 200 are temporarily affixed to the reusable frame 300, the modular, well plate system 100 can then be placed into an instrument or device common to laboratory and diagnostic practice for microplates and analogous products, such as a Molecular Devices SpectraMax, a Tecan Infinite, or a Cerillo Stratus.

Another aspect involves a novel, modular well plate system 100, comprising:
a) a plurality of disposable modules 200, each, comprising:
   i) at least one chamber; and
   ii) one or more registrations features (204, 206); and,
b) a reusable frame, comprising:
   i) at least one horizontal reference plane 104;
   ii) at least one a vertical reference plane 110;
   iii) a plurality of receiving areas circumscribed by the reference planes, each receiving area configured to receive one of the plurality of modules;
   iv) one or more registration features (108, 112) corresponding to the one or more registration features on the modules;
wherein:
   the modules are configured individually for experimental purposes;
   the modules are temporarily affixed to the frame via each module's respective registration features; and
   the frame is configured to mate with a standard multiwell plate reader and present the temporarily affixed modules for measurement.

Another aspect involves a novel, modular well plate system 100, wherein, the reusable frame 300, comprises:
a) at least one feature defining a horizontal reference plane 104;
b) at least one feature defining a vertical reference plane 110, wherein the horizontal reference plane 104 and vertical reference plane 110 together function to align the reusable frame 300 in a device that will take measurements from/of modular well(s) 200 that are present;
c) a plurality of receiving areas circumscribed by the reference planes, each receiving area configured to receive an individual module 200 and each area, further optionally comprises:
   a window or opening 302 to allow for measurement of samples contained within the individual module by the device;
d) at least one vertically oriented registration feature 112 to align each individual module 200 relative to the vertical reference plane 110; and,
e) at least one horizontally oriented registration feature 108 to align each individual module 200 relative to the horizontal reference plane 104.

In another aspect, reference planes refer to datum planes (e.g., walls) used as reference locations for the alignment of the modular well plate system (specifically the plate portion) to a desired device (or machine or instrument-used interchangeably herein).

In another aspect, examples of the plurality of receiving areas include pockets, recesses, and grooves or other features (not numbered).

Another aspect involves a novel, modular well plate system 100, wherein, the reusable frame 300, optionally comprises one or more of the following:
f) at least one registration flexure 306 configured to align an individual module relative to vertically oriented registration feature 112;
g) at least one secondary registration flexure 324 configured to align an individual module relative to horizontally oriented registration feature 108;
h) one or more module support ledges 304 configured to align and, optionally, secure individual modules 200 to the bottom surface of the frame;
i) an interior ridge 318;
j) a perimeter ridge 308, configured to work in conjunction with the interior ridge 318 to contain spills within, and either individually or in combination may be used for supporting a lid or a covering mechanism (not shown);
k) a centrally located support rib 314 or plurality of support ribs in a branching structure configured to provide mechanical support while handling the frame and affixing modules;
l) a region of recess 316 to capture a spilled sample, or serve as a basis of mechanical attachment for a lid or covering mechanism;
m) a lead-in feature 320;
n) an origin indicator 102;
o) a coordinate system to identify individual modules 106;
p) an area designated for the placement of a company logo or an inscribable plaque for writing or printing experimental information 312; and,
q) rounded corners 322 allowing the frame to fit (or more easily fit) into a variety of multi-well plate readers or similar devices.

In another aspect, the lead-in feature 320 comprises a tapered edge to aid in the placement and alignment of modules.

In another aspect, the origin indicator 102 can be used to demonstrate proper orientation for measurement and analysis.

In another aspect, the registration flexure 306 and secondary registration flexure 324 align a module to its corresponding registration feature 112 and 108, respectively, by applying a directional force. Examples of such force-producing flexures include spring tabs, precisely dimensioned features designed to create a press fit, and elastomeric components.

Another aspect involves a novel, modular well plate system 100, wherein, the one or more modular wells 200, comprise:
a) a plurality of chambers (e.g., 2, 3, 4, or more), each chamber configured to house a volume (e.g., liquid, reactants, cells, etc.), wherein the plurality of chambers are fastened or connected together (e.g., using fabrication methods common to the art);
b) at least one horizontally oriented registration feature 204 configured to align a module to a corresponding feature 108 on a reusable frame 300; and,
c) at least one vertically oriented registration feature 206 configured to align a module to a corresponding feature 112 on the reusable frame 300.

In another aspect, examples of the methods used to fasten or connect the chambers include using self-tapping screws, rivets, adhesives, laser welding, heat stamping, and ultrasonic welding.

Another aspect involves a disposable module 200, comprising: a first chamber and a second chamber, and optionally a third or more chamber. Each chamber is configured to house a volume (e.g., a first volume and a second volume). In some aspect, the plurality of volumes is fluidically connected or connected as pairs in such a way that they can be used to measure contact-independent interactions. For example, in FIGS. 3A and 3B there are two chambers illustrated (unnumbered). In an example, these two chambers comprise first and second volumes that can be used measure interactions between sets of different microbes separated by a semipermeable membrane (not shown).

Another aspect involves a novel, modular well plate system 100, wherein, the one or more modular wells 200, optionally and independently comprise one or more of the following:
d) a top surface 202 upon which lids or other types of sample coverings can optionally be attached and aligned;
e) at least one window or opening 208 to allow for measurement of samples contained within the chambers;
f) a bottom surface 210 configured to allow for the vertical positioning and retention of a module 200 by support ledges 304; and,
g) at least one alignment feature 212 used in the assembly and automation processes used to assemble the modules 202.

In another aspect, examples of the alignment feature 212 include a hole for a dowel pin to allow multiple components to align with one another during assembly.

Referring to FIGS. 1A-9, the modular well plate system 100, specifically the reusable frame 300, is optionally configured to fit a variety of module types (not shown) provided the exterior module dimensions fit into the recesses and alignment features of the reusable frame 300.

Another aspect involves a reusable frame 300 that comprises recesses for more than one type and dimensionality of disposable module (not shown). For example, a single frame could contain a mix of different sized recesses or grooves to accommodate different combinations of module types (sizes) to allow for tandem experimentation.

Referring to FIGS. 6A and 6B, one of the benefits to the present modular well plate system is that a researcher need not fill reusable frame 300 with modules to start an experiment. Only one module is needed, at least to start an experiment. More modules, even of a different type, can be added during an experiment or not at all. This is particularly useful both in reducing plastic and material waste, as well as, limiting the volumes of expensive reagents and/or other chemicals used during experimental discovery and augmentation.

Referring to FIGS. 1A and 1B, a researcher can run different types of assays and sample modules all contained in the same modular well plate system 100. Additionally, if an experiment with a given module (or modules) fails to produce a desired result, the researcher could replace that module (or modules) with another of a different type or composition.

Another aspect involves a modular well plate system wherein the module chambers match volumes and spacings of standard plate sizing. Examples of the number of module chambers include 6, 12, 24, 48, 96, and 384.

In another aspect, the number of chambers present in the modular well plate system ranges between 2-48 (or more). Examples include 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, and 48.

Another aspect involves the reusable frame configured to allow for its sterilization after use either by autoclave, ethylene oxide gas treatment, or other treatment standard to laboratory practice. As this frame and the module well plate system may be used in a sensitive environment or in an area of biological hazard, this type of design consideration would aid in the re-use of the frame component.

Another aspect involves the opacity of some or all of the walls of the disposable module 200. Fluorescence measurements typically use vessels with opaque walls (or, for example, a window in a portion of a wall) to facilitate in making an optical reading of a sample reaction as it starts to glow. For example, a module could be designed for fluorescence such that portions of it optically segregate volumes of sample such that their emission of light can be isolated and measured. Fluorescence modules could be combined into pairs with non-fluorescent samples, as an example, to measure contact-independent interactions or protein expression using different types of imaging modalities in a single frame assembly. This could help reduce variables such as timing, and environmental control during the experimental process.

Another aspect involves a module that is at least partially opaque to facilitate measurement of sample fluorescence.

Another aspect involves a module that is a well pair designed to induce contact-independent interaction between two microbial cultures.

Another aspect involves a module comprising a single well chamber with a rounded or flat bottom, e.g., in uncommon sizes or geometries that are useful to more abstract or novel sciences.

Another aspect involves a module, comprising a selective-growth substrate. For example, the module is pre-treated with the selective-growth substrate.

Another aspect involves a module that is a standard-value artificial sample for the purposes of calibration or comparison.

Another aspect involves a module, comprising a feature meant to be at a specific optical density or other parametric comparator used to calibrate a device or compare read-outs between different devices.

Another aspect involves a module that is a tissue-culture treated well designed to promote the growth of adherent cells.

Another aspect involves a module that is pre-treated with a plasma etching process or similar process to promote the growth of cells or microorganisms within and/or along the surfaces of the module. This process is sometimes referred to as tissue-culture-treatment.

Another aspect involves a module that is treated with or contains a region or feature to specifically contain or isolate an area of extracellular matrix for the purpose of differentiating and/or promoting the growth of different types of eukaryotic cells.

Another aspect involves a modular well plate system wherein the module(s) and frame are constructed from materials resistant to temperature extremes and fluctuations. This would be particularly useful in the evaluation of microbes classified as extremophiles.

Another aspect involves a modular well plate system wherein the reusable frame is configured to house a nesting version of modules that are designed to interlock or connect with each other. For example, a large module would act as a trough containing a common metabolite that other modules would nest into and be measured while constrained by both the geometry of the larger module and the reusable frame.

Another aspect involves a module, comprising: an independent light source used to conduct measurement or to promote photosynthetic growth.

Another aspect involves a module, comprising: an individual element and control component designed to positively manage the temperature of an individual module. This configuration would allow a researcher to test the optimal temperature for growing a specific culture.

All references listed herein are individually incorporated herein in their entirety by reference. Numerous modifications and variations of the present invention are possible considering the above teachings. It is therefore to be understood that within the scope of the appended claims, the invention may be practiced otherwise than as specifically described herein.

1. A modular well plate system, comprising:
a) a plurality of disposable modules, each, comprising:
i) at least one chamber; and
ii) one or more registrations features; and,
b) a reusable frame, comprising:
i) at least one horizontal reference plane;
ii) at least one vertical reference plane;
iii) a plurality of receiving areas circumscribed by the reference planes, each receiving area configured to receive one of the plurality of modules;
iv) one or more registration features corresponding to the one or more registration features on the modules;
wherein:
the modules are configured individually for experimental purposes;
the modules are temporarily affixed to the frame via each module's respective registration features; and
the frame is configured to mate with a standard multiwell plate reader and present the temporarily affixed modules for measurement.

2. The well plate system of claim 1, wherein the horizontal external dimensions of the frame match those of a standard multiwell plate.

3. The well plate system of claim 1, wherein the modules are presented at horizontal locations corresponding to the horizontal locations of one or more wells of a standard multiwell plate.

4. The well plate system of claim 3, wherein the standard multiwell plate is a 96-well plate.

5. The well plate system of claim 1, wherein the frame is sterilizable.

6. The well plate system of claim 1, wherein at least one module is a well pair designed to induce contact-independent interaction between two microbial cultures.

7. The well plate system of claim 1, wherein at least one module is at least partially opaque to facilitate measurement of sample fluorescence.

8. The well plate system of claim 1, wherein at least one module is a single, flat-bottomed sample well.

9. The well plate system of claim 1, wherein at least one module is a single, round-bottomed sample well.

10. The well plate system of claim 1, wherein at least one module is pre-treated with a selective-growth substrate.

11. The well plate system of claim 1, wherein at least one module is a standard-value artificial sample for the purposes of calibration or comparison.

12. The well plate system of claim 1, wherein at least one module is a tissue-culture treated well designed to promote the growth of adherent cells.

13. The well plate system of claim 1, wherein at least one module is treated with extracellular matrix to promote eukaryotic cell growth.

14. The well plate system of claim 1, wherein at least one module is constructed of a material resistant to the effects of extreme temperature.

15. The well plate system of claim 1, wherein at least one module is configured to nest a secondary module inside of it.

16. The well plate system of claim 1, wherein at least one module, further comprises:
a light source to conduct measurement.

17. The well plate system of claim 1, wherein at least one module, further comprises:
a light source to promote photosynthetic growth.

18. The well plate system of claim 1, wherein at least one module, further comprises:
an element designed to positively control the temperature inside that module.
